(19) United States
(12) Patent Application Publication
(10) Pub. No.: US 2011/0251454 A1
(43) Pub. Date: Oct. 13, 2011

(54) Colonoscopy Tracking and Evaluation System

(75) Inventors: Richard A. Robb, Rochester, MN (US); Gianrico Farrugia, Rochester, MN (US); William J. Sandborn, Rochester, MN (US); David R. Holmes, III, Rochester, MN (US)

(73) Assignee: MAYO FOUNDATION FOR MEDICAL EDUCATION AND RESEARCH, Rochester, MN (US)

(21) Appl. No.: 13/130,476

(22) PCT Filed: Nov. 23, 2009
(86) PCT No.: PCT/US09/65536

§ 371 Date: Jun. 28, 2011

Related U.S. Application Data
(60) Provisional application No. 61/199,948, filed on Nov. 21, 2008.

(51) Int. Cl. A61B 1/04

(52) U.S. Cl. 600/103

(57) ABSTRACT

A system for tracking and evaluating a colonoscopy procedure performed using an endoscope includes a tracking subsystem, a processing subsystem and a display subsystem. The tracking subsystem provides information representative of the location of the endoscope within a patient's colon during the procedure. The processing subsystem generates visualization metrics from images produced by the endoscope during the procedure. The display subsystem is coupled to the tracking and processing subsystems to generate a visual display of the patient's colon with information representative of the visualization metrics at associated colon locations.

### TECHNICAL FIELD

The invention relates generally to colonoscopy procedures and apparatus. In particular, the invention is a method and apparatus for tracking and evaluating a colonoscopy procedure and for providing a display representative of the visualization and evaluation in real time during the procedure.

### BACKGROUND OF THE INVENTION

Colonoscopy is the most prevalent screening tool for colorectal cancer. Its effectiveness, however, is subject to the degree to which the entire colon is visualized during an exam. There are several factors that may contribute to incomplete viewing of the entire colonic wall. These include particulate matter in the colon, subject discomfort/motion, physician attention, the speed at which the endoscope is withdrawn, and complex colonic morphology. There is, therefore, a continuing need for methods and apparatus for enhancing the visualization of the colon during colonoscopy.

### SUMMARY

The invention is a system for evaluating a colonoscopy procedure performed using an endoscope. One embodiment of the invention includes a tracking input, a video input, a processor and a display output. The tracking input receives position data representative of the location and/or orientation of the endoscope within the patient's colon during the procedure. The video input receives video data from the endoscope during the procedure. The processor is coupled to the tracking input and video input, and generates visualization metrics as a function of the video data and evaluation display information representative of the visualization metrics at associated colon locations as a function of the visualization metrics and the position data. The display output is coupled to the processor to output the evaluation display information.

### BRIEF DESCRIPTION OF THE DRAWINGS

FIG. 1 is diagram of a colonoscopy tracking and evaluation system in accordance with one embodiment of the invention.

FIG. 2 is a diagram of one embodiment of the image and signal processing that can be performed by the system shown in FIG. 1.

FIG. 3 is an illustration of one embodiment of the colon model reconstruction that can be performed by the system shown in FIG. 1.

FIG. 4 is an illustration of images processed by the system shown in FIG. 1, for evaluation of sharpness and blur.

FIG. 5 is an illustration of a video image within a colon that can be produced by the system shown in FIG. 1, with identified stool highlighted in color.

FIG. 6 is an illustration of a video image within a colon that can be produced by the system shown in FIG. 1, with the image divided into regions.

FIG. 7 is an illustration of an endoscope in accordance with the system shown in FIG. 1 within a colon, showing a range of fields of view.

FIG. 8 is an illustration of a colon and endoscope viewing vectors with respect to the endoscope centerline and endoscope path.

FIGS. 9 A and B are illustrations of a tracker in an endoscope, the system and an interface in accordance with one embodiment of the invention.

FIG. 10 is an illustration of one embodiment of a display that can be generated by the system shown in FIG. 1.

FIG. 11 is one embodiment of an image of a colon that can be generated by the system shown in FIG. 1.

### DETAILED DESCRIPTION

Enhanced colonoscopy in accordance with one embodiment of the invention includes the combination of magnetic or other tracking technology, video data from the colonoscope, and signal processing software. The use of enhanced colonoscopy identifies regions of the colon that may have been missed or inadequately viewed during an exam. The addition of data from a preceding CT colography scan (if one was performed) is incorporated in other embodiments, and would provide additional benefit when available. Any pre-acquired data can be used for this purpose, including CT, MR or Nuclear Medicine scan to provide structural information (e.g., the shape of the colon) or functional information (e.g., potential lesions). The software would use the CT colography data to inform the colonoscopist when the endoscope is approaching a lesion identified on CT colography. However, since CT colography increases costs and limits this enhancement procedure to fewer clinical sites and cases, the system will guide the endoscopist to achieve nearly 100% viewing of the colon without the requirement for a CT scan prior to the procedure. The invention can be integrated into existing colonoscopy systems from multiple manufacturers or implemented as a stand-alone system.

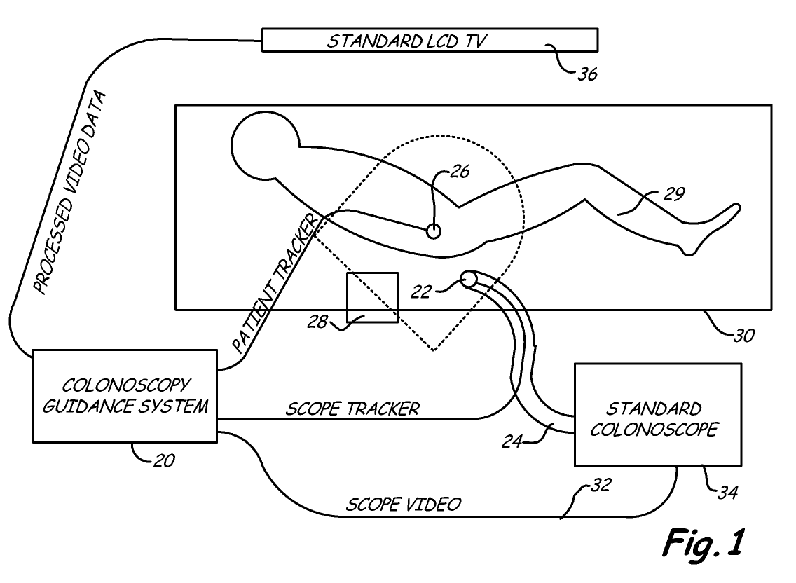

During the procedure, a tracked scope is connected to the colonoscope computer as well as to an external computer system which collects the tracking and video data. FIG. 1 is a diagram of the acquisition system. The illustrated embodiment of guidance system 20 has 4 inputs and one output. One input is from the scope tracker(s) 22. The trackers 22 may be introduced through the access port of the endoscope 24 to the tip of the scope, integrated into the scope, or attached via a large “condom” type of sleeve over the scope (not shown). Another input is from a patient reference tracker 26 that is taped to the patient 29. A magnetic reference 28 is attached to the patient table 30 in close proximity to the patient in order to generate a magnetic field signal which the tracker system uses to determine the position of the scope 24 and patient 29 via reference tracker 26 during the procedure. An endoscope video cable 32 is connected from the output of the standard colonoscopy system 34 to a digitizer card located in the guidance system 20. The guidance system 20 processes the data in real-time (or with sufficiently low latency to provide timely information) and generates a processed video data stream which is connected to a standard LCD TV 36 or other display found in most (if not all) colonoscopy suites. Other embodiments (not shown) use alternative tracking technologies including mechanical tracing (e.g., shape tape) and imaging (e.g., fluoroscopy).

The endoscopist conducts the colonoscopy in a routine manner using the standard LCD TV 36. The guidance system 20 can record and process both the scope position and video data and generate a visualization which will approximately represent the colon in 3D and provide feedback about regions of the colon which have been missed or poorly viewed. The display can be generated in real time or otherwise sufficiently fast to enable the endoscopist to utilize the information from the display without disturbing normal examination routine. Other display approaches that provide the visualization information described herein can be used in other embodiments of the invention.

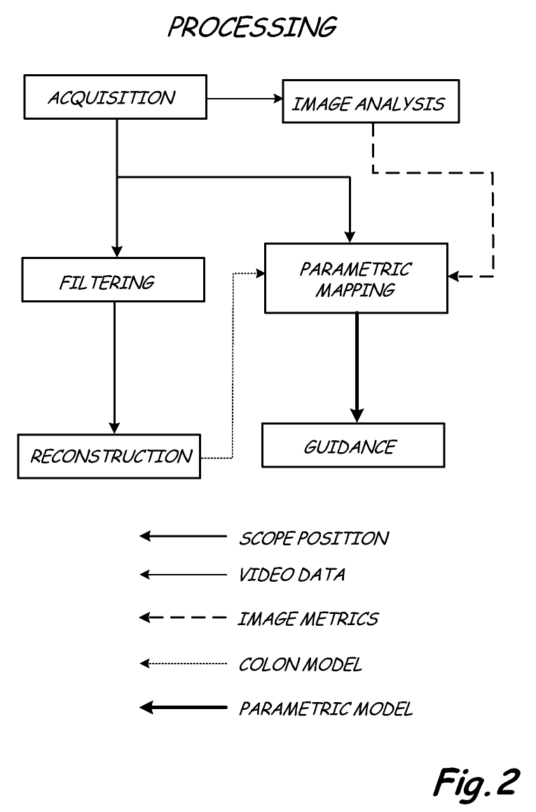

There are several technical components in this approach which can coordinate the tracker data and video data. These include (1) reconstructing the colon centerline and endoluminal surface, (2) mapping video data properties to the reconstructed colon, (3) evaluating the quality of the video data stream, and (4) presenting the data in a manner which can guide the endoscopist to examine missing or poorly viewed regions of the colon. FIG. 2 is a flow chart of one embodiment of the image and signal processing approaches that can be used with the invention. Other embodiments can use other approaches.

Each processing component of the described embodiment uses a common notation described below:

Fₜ video frame acquired at time t

IMₜ a vector of image metrics (1, 2, . . . , N) for frame Fₜ

refₜ a sampled 3D position (x, y, z) from the ref. patch at time t

scopeₜ a sampled 3D position (x, y, z) from scope at time t—

Pₜ a patient-corrected position of the scope computed from scopeₜ and refₜ

{P} the ordered point collection following filtering

{ P̅} the ordered set of all points collected

{C} the ordered set of all points in the centerline

M the ordered set of verticies and corresponding edges in the 3D colon model

{ P̅′} the ordered set of sampled points projected onto the centerline

During acquisition, three coordinated signals are acquired—the video frame (Fₜ), the position of the scope tip (scopeₜ), and the position of the reference patch (refₜ) located on the patient's back. The patient tracker position is subtracted from the endoscope tracker position to yield a patient-corrected position of the scope, Pₜ. This ensures that any gross patient motion is not characterized as endoscope motion. Since the magnetic reference is attached to the table, table motion is not a problem because its position relative to the magnetic reference is fixed. Processing begins when there are a predetermined number of points collected in the set ({P}) which can range from a small number of points to the entire path traversed by the scope. Other embodiments (not shown) making use of multiple tracker points acquired at a single time point (e.g., from multiple sensors or an imaging method such as fluoroscopy) can use a similar methodology. In embodiments such as these the subscript “t” can be replaced by the subscript “n” referring to an ordered sample of points collected at one time rather than across time.

The set of patient-corrected scope position points may require filtering to reduce noise depending on the quality of the tracked data. Both linear and non-linear filtering methods can be used alone or in combination depending on the type of noise present.

Linear filtering can be used to uniformly remove high frequency noise (such as system noise from the tracker). A moving average filter of size N may be implemented as:

$$\{ \overline{P} \} = \{ {\overline{P}}_{t} \text{:} {\overline{P}}_{t} = \frac{1}{N} {\sum }_{j = 0}^{t + N + 1} {P}_{j} \}$$

Non-linear filtering can be used to remove spurious noise from the data in which single samples are well-outside of specification. For example,

$$\{ \overline{P} \} = \{ {\overline{P}}_{t} \text{:} {\overline{P}}_{t} = \{ \begin{matrix}{P}_{t} - 1 & \vert \vert > threshold \\ {P}_{t} & else\end{matrix} \}$$

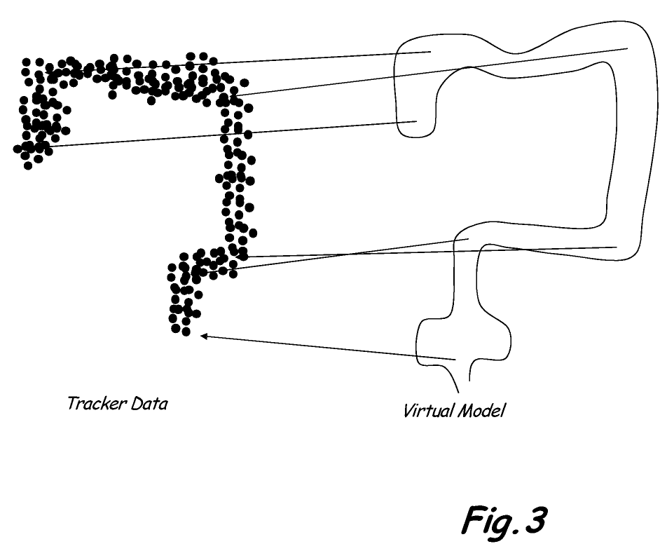

The purpose of reconstruction is to use the collected points to generate an approximate model of the colon based on the position of the scope during an exam. This is illustrated in FIG. 3. Through this process, the method generates a centerline of the colon ({C}) which is needed in subsequent processing. In one method the centerline can be created from a pre-defined model or a model can be created from a pre-defined centerline.

When using a pre-defined centerline, the centerline, {C}, can be approximated from the sampled scope positional data. There are several approaches for generating a centerline including:

One-to-One Mapping of { P̅}: The filtered points can be used directly as an approximation of the centerline ({C}={ P̅}).

Spline-fitting: Splines may be used to reduce the number of points in { P̅} while smoothing as well.

Statistical centerline calculation: In this approach, the center-line is calculated from a statistical volume created from { P̅}. One such approach to create a statistical volume is through a parzen windows function

$$PW ( \{ \overline{P} \} , σ ) = {\sum }_{t = 1}^{T} gaussian ( {\overline{P}}_{t} , σ )$$

The resulting volume provides a likelihood map of the location of the interior of the colon. The map can be thresholded to generate a mask of where the scope has traveled, defining the interior of the colon. A shortest path method can be used to generate the centerline from the mask.

Once the centerline is created, a model can be generated, for example, by extruding a primative shape along the points in {C}. In one implementation of this model, the primative is defined as a discrete set of ordered points at a fixed radius (r) which describe a circle

{circle}={(x,y):x=r·cos(0 . . . 2π),y=r·sin(0 . . . 2π)}

and the extruded model is

M={Cₜ:Cₜ=T·circle

where T is the transformation matrix defined by the (Cₜ−Cₜ₋₁)}

When using a pre-defined model of the colon, the model of the colon can be fit to the tracking data. The pre-defined model is deformed to fit the tracker data. To account for soft tissue deformation, the virtual model can be “pliable” in the virtual sense such that it can be stretched or twisted to fit the tracker data. Either a patient-specific virtual model or a generic anatomic virtual model can be used to register the tracker data. This fitting task would initialize the pre-determined model (and its corresponding centerline {C})—which can be derived from pre-existing generic data or the patient's image data—in the space of { P̅}.The task to align the pre-defined model with the positional data { P̅}, can be achieved with several methods including, landmark and surface fitting.

Using landmark fitting, anatomical landmarks (or specific regions of the colon) such as the appendiceal orifice and ileocecal valve in the cecum, the hepatic flexure, the triangular appearance of the trans-verse colon, the splenic flexure, and the anal verge at the lower border of the rectum can be used to align specific points ( P̅ₜ) from { P̅} with corresponding points in the model.

Using surface fitting, the pre-determined model can be deformed (with or without constraints) such that it maximizes the number of P̅ₜ from { P̅} which fall within the interior of the model.

Following reconstruction, the model (M) and corresponding centerline ({C}) are used for mapping the original points {P} into the model.

Alternatively or in addition, the tracker data can be used to compute an approximation of the centerline of the colon. After the computed centerline is generated, a generic surface can be created with a circular cross section having a fixed radius. While these approaches may not specifically reconstruct the exact true geometry of the colon, the true surface geometry is not required for guiding the procedure in accordance with the invention.

Any of a number of image quality metrics (represented as vector IMₜ) can be determined from the video data. These include intensity, sharpness, color, texture, shape, reflections, graininess, speckle, etc. To realize real-time processing with the system, metrics can be approximated or sparsely sampled for computational efficiency. Intensity, for example, may serve as a useful metric of quality—darker regional intensity is a lower quality region whereas higher regional intensity is better image data. Regional sharpness, calculated as

$$as σ ( \frac{d ( I )}{d x} ) + σ ( \frac{d ( I )}{d y} ) / 2 ,$$

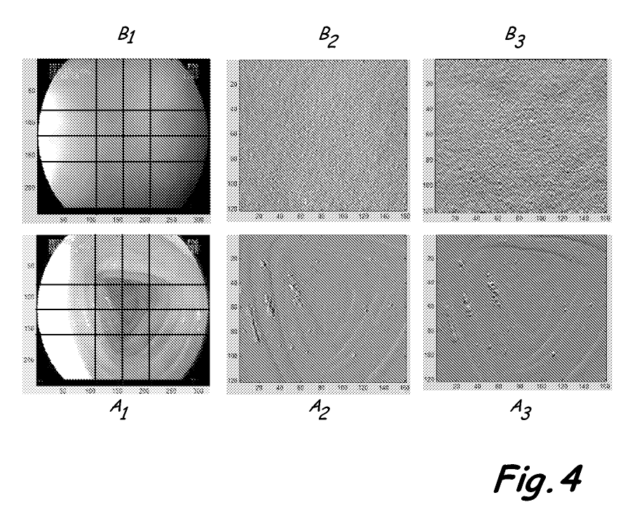

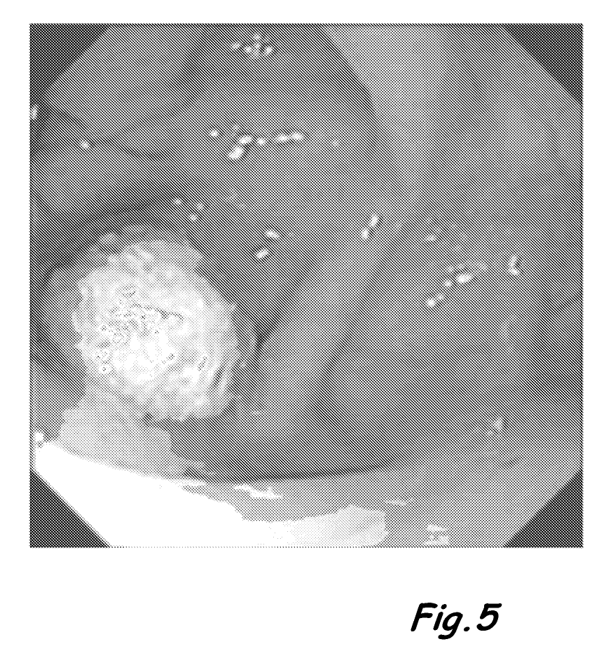

can be used to determine the quality of the image data—higher sharpness is less blurry data. In FIG. 4, the regional sharpness is high in the image A1 which is indicative of a better image. Color characterization can be used to identify stool in the field of view. FIG. 5 shows stool highlighted in yellow and green. Such color differences can be determined and characterized by multi-spectral analysis methods. Foam, which is sometimes seen in the field of view, can be characterized either by color or texture. Texture and shape (as estimated from edge curvature within the image) can be used to classify abnormalities or pathology. Multispectral analysis of combinations of these image features can potentially add to the robustness of image quality classification.

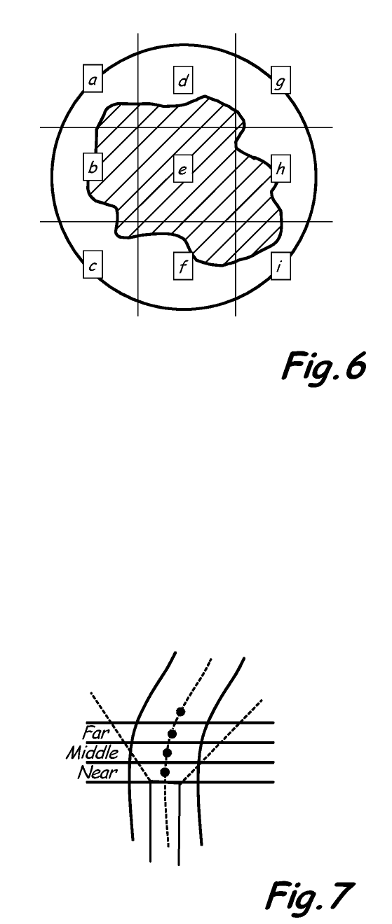

Analysis of regions of interest (ROIs) can be used to further refine the image classification of quality analysis. For example, each video image can also be partitioned into nine regions a-i as shown in FIG. 6. Each region is evaluated based on image intensity using the assumption that the far field is darker than the near field. Together, the intensity regions can be used to determine the direction of viewing along with depth of viewing. For example, if regions a, b, & c, are dark while regions g, h, & i, are bright, it suggests that the camera is pointed right with a, b, & c, in the far field. While an arbitrary number of field depths can be defined, three can provide adequate fidelity for mapping video quality—the near field, middle field, and far field. The difference, in this case, is that far field data is likely to be too dim to view adequately; the near field may be too close and therefore blurry; the preferred viewing distance is the middle field in one embodiment. As quality of video data is calculated, each region will map the processed data to centerline points at the tip of the scope (near field), a small distance out (middle field), or a long distance away (far field). It is expected that most of the data at the near and far field will be of lower quality. FIG. 7 shows the near, middle, and far fields, associated with their corresponding centerline positions.

The fusion of the model, original data, and results of the video data constitute the parametric mapping component. In preparation for mapping the video data onto the virtual model, the tracker data is normalized to the centerline of the colon to generate “standard views” from the scope. The benefit is that if the same section is viewed multiple times from different angles, the corresponding “standard view” will be the same.

The patient tracker position can be subtracted from the endoscope tracker position to ensure that any gross patient motion is not characterized as endoscope motion. Since the magnetic reference is attached to the table, table motion is effectively eliminated because the table position relative to the magnetic reference will not change. Each endoscope tracker point can be mapped to the pre-defined centerline by determining the closest centerline point to the vector defined by the tracker data. Accordingly, if the endoscope doesn't move, but looks to sides such as left or right, then all the acquired video frames will be associated with the same centerline point, but at different viewing angles.

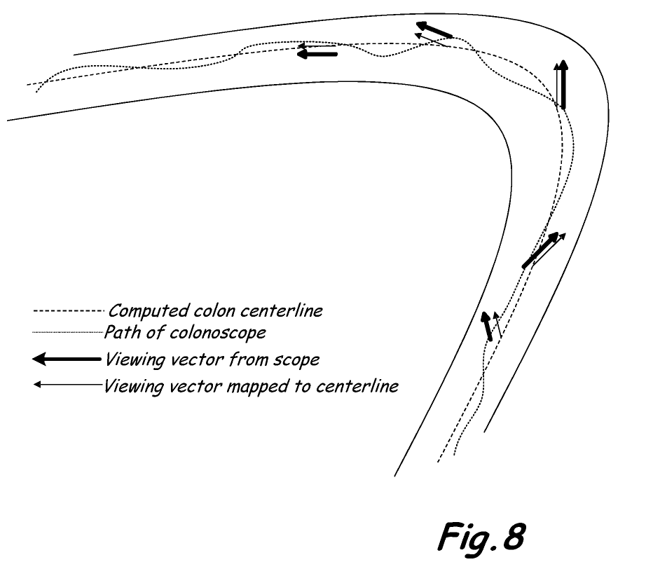

The mapping is as follows in one embodiment of the invention, although other approaches can be used. Each point of the originally sampled points ( P̅ₜ) is projected to a point along the centerline ({ C̅}). This is calculated as the point on the centerline which is the minimum distance to each P̅ₜ. FIG. 8 illustrates this step. The metric vector, IMₜ computed from Fₜ, is then stored with its corresponding projected point qₜ. Since multiple frames will likely be projected to the same qₜ, the metrics may be aggregated together:

IM′ₜ=aggregate(IMₜ at qₜ)

where the aggregate function may be an average, max, min, median, or other functions. Using a pre-defined color scale, the {IM″ₜ} set is then used to color onto the surface of the M at each vertex.

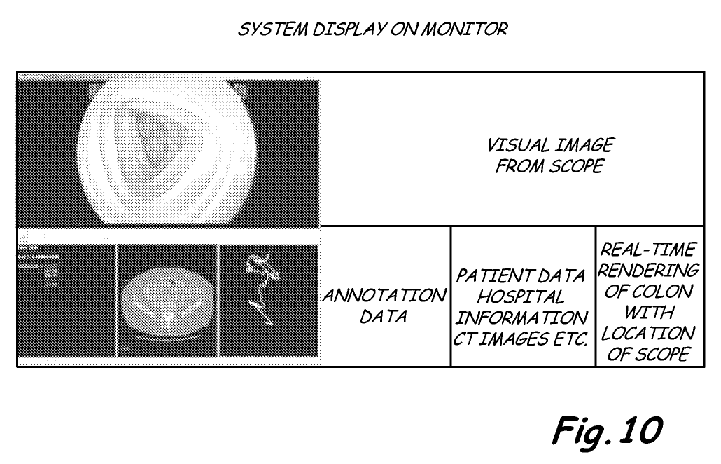

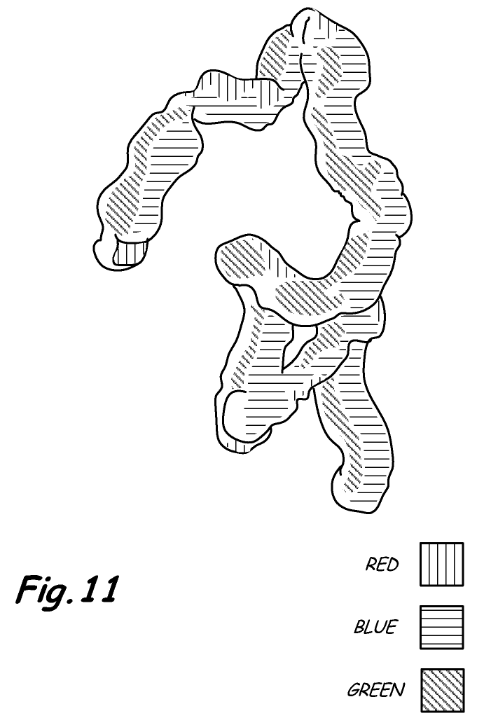

Presentation of the processed signal and image data is primarily driven by the virtual model of the colon. The model provides an approximate, patient-specific, representation of the colon. On the surface of the colon, color patches are displayed to identify regions of high and low quality data. The patch color can vary according to a pre-defined color scale. White might be used in regions of the colon that have not been viewed at all. Red regions might suggest that only low quality images have been collected whereas green patches may show regions of high quality images (free of stool and foam, sharp images with adequate lighting and color). FIG. 11 is an example of a colon image generated by the method and system of the invention, with red areas showing regions of low-quality images, green areas showing regions of high-quality images, and blue areas showing regions of the colon with no visual confirmation of viewing based on the video. In addition, the intensity of the color patches can be used to indicate the number of frames viewed at that position in the colon. Further, sub-regional analyses can display the color patches radially distributed around the centerline position. The virtual model may be built using any subset of sample points, however, it is advantageous in some embodiments to build the model during insertion and used to guide during removal. FIG. 10 is an illustration of a display that can be presented on the LCD TV. During review of the virtual model, previously acquired video frames can also be displayed for review.

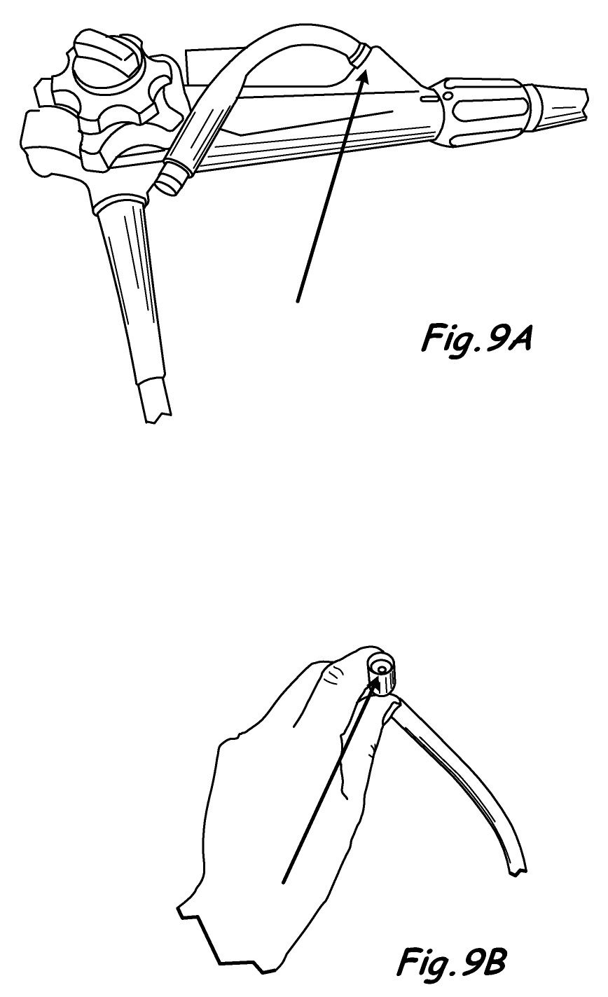

In one embodiment, the system is implemented on a mobile cart which can be brought into a procedure room prior to the start of a colonoscopy. Other versions can be fully integrated into the procedure room. FIG. 9 shows one embodiment of the tracker in an endoscope, the entire system, and the interface. In this case, the computational component is a multi-core computer (e.g., Quad-core Dell computer) with large amounts of memory and disk. A medium-ranged magnetic tracker (e.g., Ascension Technologies MicroBird tracker) is used for tracking both the endoscope and patient. The transmitter is attached to a stand which is attached to the patient table during a procedure. The system contains a high end video capture card (e.g., EPIX systems) which acquires all of the data from the colonoscopy system. The tracking sensors on the scope can be hardwired or made wireless. There can be one or more sensors along the shaft of the scope. Multiple sensors along the shaft of the scope can be used to detecting “looping” of the scope/bowels during insertion. The sensors can be attached/embedded within a sleeve or condom to retrofit the sensors to any current scope.

In one embodiment, the software is a multi-threaded application which simultaneously acquires both the tracker data and video data in real-time. In addition to storing all of the data to disk, the data is processed in real-time and drawn to the screen. The same display is also sent to the LCD TV in the procedure room.

The invention can be performed using segmental analysis. In this embodiment, the colon will be divided into segments. These segments can include, but not be limited to, the cecum, proximal to mid ascending colon, mid ascending to hepatic flexure, hepatic flexure, proximal to mid transverse colon, mid transverse to splenic flexure, splenic flexure, proximal descending to mid descending, mid descending to proximal sigmoid, sigmoid, and rectum. Each segment can be visualized at least twice and the data images analyzed and compared to determine the degree of visualization. For example a concordance between sweeps 1 and 2 of 100% can be interpreted as to mean that 100% of the mucosa was visualized, while a lower level of concordance may indicate ever decreasing visualization rates. These data sets will be computed in real time or near-to-real time and the information provided in a variety of means, including visual and/or auditory in order to inform the proceduralist of the results and aid in decision making regarding adequate visualization of the mucosa.

Prior exam data can be incorporated into other embodiments of the invention. For example, prior examination data from two sources can be used. One source of prior data is pooled data from multiple endoscopists. This data could provide a statistical likelihood and 95% CI (confidence interval) that the mucosa in a given segment of the colon has been visualized with blur free images. Data used to provide this instrument could include examinations where mucosal surface visualized has been verified by more than one examiner, or by correlation with another technology such as CT colonography. Other relevant data that might modify the likelihood can include the speed of withdrawal, the specific anatomic segment (variable likelihood in different segments), the number of times the segment has been traversed, etc. The second source of prior data is examinations from the specific endoscopist. Endoscopist specific modifiers of the likelihood of complete mucosal visualization could include the speed of withdrawal, and perhaps even some seemingly unrelated factors like the specific endoscopist's overall polyp detection rate, etc. (i.e. some endoscopists might need more of an accuracy handicap than others).

Relevance feedback can also be incorporated into the invention. In embodiments including this feature, information provided by the computer system is tailored to be non-disruptive yet compulsive in indicating the extent and quality of visualization within a temporal and/or spatial block. This is achieved through a relevance feedback framework wherein the system gauges the efficacy of its extent/quality cues as a function of the endoscopist's subsequent response and uses this information to iteratively achieve an improved cueing subsequently.

The system provides extent/quality cues to the recently visualized segment and objectively interprets the subsequent actions of the endoscopist as to whether, and to what degree, the cues are relevant or irrelevant to the exam. The system then learns to adapt its assumed notion of quality and or coverage to that of the endoscopist. The feedback operates in both greedy and cooperative user modes. In the greedy mode, the system provides feedback for every recently visualized region. In the cooperative user mode wherein a segment is repeatedly visualized in multiple sweeps, the feedback progressively learns, unlearns and relearns its judgment.

Computational strategy for achieving relevance feedback involves “active learning” or “selective sampling” of extent/quality-sensitive features, in-order to achieve the maximal information gain, or minimized entropy/uncertainty in decision-making. Active learning provides accumulation, stratification and mapping of knowledge during examination from time to time, segment to segment, endoscopist to endoscopist and from patient to patient. Resultant mapping learned across the spectrum can potentially minimize intra-exam relevance feedback loops which might translate into an optimal examination.

An accelerometer can also be incorporated into embodiments of the invention described herein. An accelerometer embedded at or near the tip of the colonoscope, for example, will provide feedback regarding the motion of the scope. In particular, the “forward” and “backward” motion of the scope provides useful information about the action of the endoscopist. “Forward” actions (in most but not all cases) are used during insertion to feed the scope through the colon; “backward” motion (in most cases but not all) is the removal of the scope and is often associated with viewing of the colon. For the purposes of computer assisted guidance, the path of the scope path may be constructed during insertion only, whereas image analysis may occur during removal. Alternatively, multiple forward and back motions may indicate direct interrogation of folds or other motions which would confound the automated analysis; this could be determined from the accelerometer data. Additional accelerometers can be populated along the length of the scope. Using a flexible tube model, the combination of accelerometers can be used to infer some features of the shape of the scope. In particular, multiple adjacent sensors could be used to detect looping of the scope. Moreover, during insertion or pullback, the repeated capture of multiple accelerometers can be used to reconstruct the path of the entire scope. An inertial navigation system (INS)—generally a 6 DOF (degree of freedom) measurement device containing accelerometers and gyroscopes—can also provide local motion estimates and be combined with other INS devices to infer features of the entire scope including the shape of the scope.

A stereoscopic view/laser range finder can be incorporated into the invention. Reconstruction of the local 3D geometry can be achieved through several different methods. A combination of stereo views and image processing (texture/feature alignment) can be used to reconstruct the 3D geometry from a scene. Stereo optics can, for example, be incorporated into the colonscope. Alternatively, a specialty lens could be attached to the tip of a scope to achieve a stereoscopic view. This can be achieved through a lenticular lens or possibly multiple lenses which are interchangeably placed in front of the camera. A visible light filter can be swept across the scene to reconstruct the 3D surface (in a manner similar to laser surface scanners and/or laser range finders). A combination of multiple views from a tracked camera can also be used to reconstruct the interior surface of the colon. The reconstructed 3D surface can be used to detect disease such as polyps (based on curvature), evaluate normal, abnormal, and extent of folding of the colon wall, and precisely measure lesion size.

Insufflation can also be used in connection with the invention. Poor insufflation of the colon results in poor viewing of the colon wall (particularly behind folds). Automatically determining the sufficient insufflation is an important process to incorporate in the system. Using a 3D surface reconstruction system the uniformity of the colon wall can be used as a metric for proper insufflation. The extent of folds can also be estimated from the video data. Specifically, local image features such as the intensity gradient can be used to determine the shape and extent of folds within the field of view. Finding a large number of image gradients located in close proximity suggests a fold in the colon wall. Alternatively, by varying the insufflation pressure slightly, the changes in image features (such as gradients) can provide an estimate of fold locations and extent of folds.

Although the present invention has been described with reference to preferred embodiments, those skilled in the art will recognize that changes can be made in form and detail without departing from the spirit and scope of the invention.

1. A system for tracking and evaluating a colonoscopy procedure performed using an endoscope, including:
a tracking subsystem to provide information representative of the location of the endoscope within a patient's colon during the procedure;
a processing subsystem to generate visualization metrics from images produced by the endoscope during the procedure; and
a display subsystem coupled to the tracking and processing subsystems to generate a visual display of the patient's colon with information representative of the visualization metrics at associated colon locations.

2. The system of claim 1 wherein the visualization metrics include image quality metrics.

3. The system of claim 2 wherein the image quality metrics include regional sharpness.

4. The system of any of claim 1 wherein the visualization metrics includes colon substance (e.g., stool and/or foam) metrics.

5. The system of any of claim 1 wherein the visualization metrics includes one or more of intensity analysis, depth of view analysis and direction of view analysis.

6. The system of any of claim 1 wherein the visualization metrics includes metrics vectors such as distance, size, shape and texture.

7. The system of any of claim 1 wherein the endoscope location information is normalized to the colon centerline.

8. The system of any of claim 7 wherein:
the system further includes a colon model subsystem to generate a model of the colon as a function of the endoscope location information; and
the display subsystem generates a visual display of the model of the colon generated by the colon model subsystem.

9. The system of claim 8 wherein the colon model system generates the colon model using one of a predefined model and an predefined centerline.

10. The system of any of claim 1 wherein the visual display of the patient's colon is color coded to represent the visualization at the associated colon locations.

11. The system of any of claim 1 wherein the visual display of the patient's colon includes information representative of the amount of video viewed at the location of the colon.

12. A system for evaluating a colonoscopy procedure performed using an endoscope, including:
a tracking input for receiving position data representative of the location and/or orientation of the endoscope within the patient's colon during the procedure;
a video input for receiving video data from the endoscope during the procedure;
a processor coupled to the tracking input and video input, generating visualization metrics as a function of the video data and generating evaluation display information representative of the visualization metrics at associated colon locations as a function of the visualization metrics and the position data; and
a display output coupled to the processor for outputting the evaluation display information.

13. The system of claim 12 wherein the visualization metrics generated by the processor include image quality metrics.

14. The system of claim 12 wherein the visualization metrics generated by the processor include colon substance metrics.

15. The system of claim 12 wherein the visualization metrics generated by the processor include one or more of intensity analysis, depth of view analysis and direction of view analysis.

16. The system of claim 12 wherein the evaluation display information generated by the processor includes information representative of a visual display of a colon model and visualization metrics at associated locations on the colon model.

17. The system of claim 16 wherein the processor generates the evaluation display information in real time or near real time during the procedure.
